(19) United States
(12) Patent Application Publication
(10) Pub. No.: US 2006/0009470 A1
(43) Pub. Date: Jan. 12, 2006

(54) Method for specifically potentiating N-type Ca2+ channel activity

(75) Inventors: Shuji Kaneko, Kyoto-shi (JP); Nobuya Matsuoka, Osaka-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: Shuji KANEKO, Kyoto-shi (JP); Fujisawa Pharmaceutical Co., Ltd., Osaka-shi (JP)

(21) Appl. No.: 11/228,325

(22) Filed: Sep. 19, 2005

Related U.S. Application Data
(62) Division of application No. 10/084,160, filed on Feb. 28, 2002.

(30) Foreign Application Priority Data
Aug. 28, 2001 (JP) 2001-258808

(51) Int. Cl. A61K 31/4965

(52) U.S. Cl. 514/255.01

(57) ABSTRACT

The present invention provides a method of specifically potentiating an N-type Ca²⁺ channel activity, which includes administering, to a subject, a compound of the following formula (I): 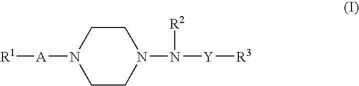
wherein R¹ is lower alkyl, aryl, ar(lower)alkoxy or a heterocyclic group, the above groups being optionally substituted by halogen, R² is hydrogen atom or lower alkyl, R³ is cyclo(lower)alkyl, aryl or ar(lower)alkyl, the above groups being optionally substituted by halogen, A is —CO—, —SO₂— or lower alkylene, and Y shows —CO—, —SO₂— or —CONH— (compound (I)), a salt thereof, a prodrug thereof or a solvate thereof; a method for the prophylaxis or treatment of brain disorders such as dementia and amnesia, which includes such administration; and a screening method particularly useful for screening a compound having a similar effect as compound (I).

### TECHNICAL FIELD OF THE INVENTION

The present invention relates to a method for specifically potentiating an N-type Ca²⁺ channel activity. More particularly, the present invention relates to a method for the prophylaxis or treatment of brain disorders, which comprises administering an effective amount of a compound having an effect of specifically potentiating an N-type Ca²⁺ channel activity to patients. The present invention further relates to a screening method of a compound having an effect of specifically potentiating an N-type Ca²⁺ channel activity and a method for the prophylaxis or treatment of brain disorders, which comprises administering an effective amount of a compound obtained by such a screening method to patients.

### BACKGROUND OF THE INVENTION

The calcium channel (also referred to as a Ca²⁺ channel in the present specification) is an ion channel that selectively allows permeation of a calcium ion and includes a voltage-dependent calcium channel and other calcium channels. The voltage-dependent calcium channel is a calcium channel that opens on depolarization of a cell membrane potential, and is widely distributed in excitable cells of nerves, muscles, secretory cells and the like. The calcium channel is divided into L-type, N-type, P-type, T-type and the like, based on voltage-dependency, activation-inactivation rates, tissue distribution and differences in pharmacological properties. The calcium channel consists of several subunits, inclusive of α₁ subunit responsible for the function as a channel, selectivity to calcium, voltage-dependency and the like, and other subunits (e.g., β subunit and α₂δ subunit in N-type calcium channel) considered to modify the function.

Hippocampus is a brain region located in the interior of the temporal lobe, and controls learning and memory. When the input neuron of hippocampus is stimulated at high frequency in a short time, a phenomenon where synaptic transmission efficiency keeps increasing for a long time thereafter is observed. This phenomenon is called a long-term potentiation of synaptic transmission (hereinafter to be also referred to as LTP), and is recognized to be a cellular model of learning and memory (T. V. P. Bliss and G. L. Collingridge, Nature, 361, 31 (1993)). Further elucidation of the mechanism of LTP and the relationship between the mechanism and learning/memory has been desired.

WO 00/72834 discloses that a compound having the following structure (I) (hereinafter to be also referred to as compound (I)) promotes release of somatostatin in brain and expresses a synaptic transmission long term potentiating effect, for which the compound can be used as a therapeutic agent for dementia and the like: 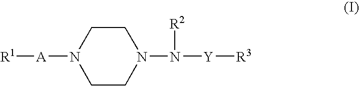
wherein R¹ is lower alkyl, aryl, ar(lower)alkoxy, or a heterocyclic group, the above groups being optionally substituted by halogen, R² is hydrogen atom or lower alkyl, R³ is cyclo(lower)alkyl, aryl or ar(lower)alkyl, the above groups being optionally substituted by halogen, A is —CO—, —SO₂— or lower alkylene, and Y shows —CO—, —SO₂— or —CONH—.

The compound (I) shows a memory and learning improving effect and an LTP-promoting effect in the hippocampus CA3 region. The mechanism of LTP promotion by compound (I) is suggested to involve activation of somatostatinergic neurotransmission. In addition, compound (I) has been found to gradually increase population spike amplitude (PSA) in the perforating fiber-dentate gyrus synapse of hippocampus slice. However, the mechanism by which compound (I) exhibits the above-mentioned effect has not been clarified.

It is therefore an object of the present invention to develop a novel screening method of a compound useful for the development of an agent for the prophylaxis or therapy of brain disorders and to provide a novel medicament using this screening method.

### SUMMARY OF THE INVENTION

According to the present invention, it has been found that an action on an α₁ subunit of a particular Ca²⁺ channel potentiates the channel-specific current, thereby promoting inflow of Ca²⁺, and shows an effect useful for the prophylaxis or treatment of brain disorders, and further that a compound useful as an agent for the prophylaxis or treatment of brain disorders can be screened using, as an index, changes in the cell membrane current of a cell made to express the subunit. Based on the findings, the present inventors have established the method therefor and completed the invention. Accordingly, the present invention provides the following.
[1] A method for specifically potentiating an N-type Ca²⁺ channel activity, which method comprises administering an effective amount of a compound of the following formula (I): 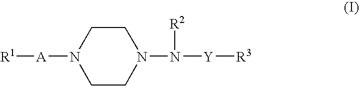
   wherein R¹ is lower alkyl, aryl, ar(lower)alkoxy or a heterocyclic group, the above groups being optionally substituted by halogen, R² is hydrogen atom or lower alkyl, R³ is cyclo(lower)alkyl, aryl or ar(lower)alkyl, the above groups being optionally substituted by halogen, A is —CO—, —SO₂— or lower alkylene, and Y shows —CO—, —SO₂— or —CONH—, a salt thereof, a prodrug thereof or a solvate thereof to a subject.
[2] The method of the above-mentioned [1], wherein the compound of the aforementioned formula (I) is N-(4-acetyl-1-piperazinyl)-p-fluorobenzamide monohydrate.
[3] A method for the prophylaxis or treatment of brain disorders, which comprises administering an effective amount of a compound having an effect of specifically potentiating an N-type Ca²⁺ channel activity to a subject.
[4] The method of the above-mentioned [3], wherein the aforementioned brain disorder is selected from the group consisting of dementia, amnesia, schizophrenia, manic-depressive psychosis, stroke, head trauma, nicotine withdrawal symptom, spinal trauma, anxiety, thamuria, incontinence of urine, myotonic dystrophy, attention deficit hyperactivity disorder, narcolepsy, Parkinson's disease, autism and psychosomatic disorder.
[5] The method of the above-mentioned [3] or [4], wherein the compound having an effect of specifically potentiating an N-type Ca²⁺ channel activity is a compound of the following formula (I): 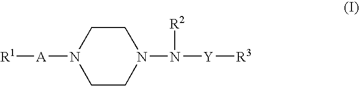
   wherein R¹ is lower alkyl, aryl, ar(lower)alkoxy or a heterocyclic group, the above groups being optionally substituted by halogen, R² is hydrogen atom or lower alkyl, R³ is cyclo(lower)alkyl, aryl or ar(lower)alkyl, the above groups being optionally substituted by halogen, A is —CO—, —SO₂— or lower alkylene, and Y shows —CO—, —SO₂— or —CONH—, a salt thereof, a prodrug thereof or a solvate thereof.
[6] The method of the above-mentioned [5], wherein the compound of the aforementioned formula (I) is N-(4-acetyl-1-piperazinyl)-p-fluorobenzamide monohydrate.
[7] A method for screening a compound having an effect of specifically potentiating an N-type Ca²⁺ channel activity, which method comprises steps of bringing a neuronal voltage-dependent calcium channel α_{1B} subunit expression cell into contact with a test compound; measuring a membrane current of the cell; bringing a neuronal voltage-dependent calcium channel α_{1B} non-expression cell into contact with a test compound; measuring a membrane current of the non-expression cell; and comparing the membrane current of the aforementioned expression cell and the membrane current of the non-expression cell.
[8] The method of the above-mentioned [7], wherein the aforementioned neuronal voltage-dependent calcium channel α_{1B} non-expression cell is a cell made to express a neuronal voltage-dependent calcium channel α_{1A} or α_{1E}.
[9] The method of the above-mentioned [7] or [8], wherein the aforementioned expression cell is Xenopus oocyte made to express a neuronal voltage-dependent calcium channel α_{1B} subunit.
[10] The method of any of the above-mentioned [7] to [9], wherein the aforementioned neuronal voltage-dependent calcium channel α_{1B} non-expression cell is Xenopus oocyte made to express a neuronal voltage-dependent calcium channel α_{1A} or α_{1E}.
[11] The method of the above-mentioned [3], wherein the compound having an effect of specifically potentiating an N-type Ca²⁺ channel activity is obtained by the screening method according to any of the above-mentioned [7] to [10].

### BRIEF DESCRIPTION OF THE DRAWINGS

FIG. 1 shows an effect of N-(4-acetyl-1-piperazinyl)-p-fluorobenzamide monohydrate on α_{1A}, α_{1B} and α_{1E} channel currents, wherein shown are tracks of the currents before application of N-(4-acetyl-1-piperazinyl)-p-fluorobenzamide monohydrate (basal), upon application of 100 nM N-(4-acetyl-1-piperazinyl)-p-fluorobenzamide monohydrate (90 seconds after contact of a medicament solution with cell), and 30 seconds after washing.

FIG. 2 is a graph showing the relationship between concentration and response in the effect of N-(4-acetyl-1-piperazinyl)-p-fluorobenzamide monohydrate on the Ca²⁺ channel of a nerve cell.

### DETAILED DESCRIPTION OF THE INVENTION

In the present invention, by the “effect of specifically potentiating an N-type Ca²⁺ channel activity” is meant an action (significant changes from the baseline) to potentiate the current of an N-type calcium channel, which is a neuronal voltage-dependent channel. It refers to the ability to induce a statistically significant level of potentiation at a certain concentration without exerting a statistically significant influence on different types of channels, when measured using a method of, for example, two electrode membrane voltage-clamp method and the like.

In the present invention, by the “brain disorder” is meant dementia (e.g., dementia caused by various pathosises such as senile dementia, Alzheimer's dementia, cerebrovascular dementia, dementia after cerebral trauma, dementia caused by cerebral tumor, dementia caused by chronic subdural hematoma, dementia caused by normal pressure hydrocephalus, dementia caused by meningitis, Parkinsonian dementia and the like), amnesia, schizophrenia, manic-depressive psychosis, stroke, head trauma, nicotine withdrawal symptoms, spinal trauma, anxiety, thamuria, incontinence of urine, myotonic dystrophy, attention deficit hyperactivity disorder, narcolepsy, Parkinson's disease, autism, psychosomatic disorder and the like.

Specific examples of the compound having such effect include compounds (I), wherein N-(4-acetyl-1-piperazinyl)-p-fluorobenzamide monohydrate is particularly preferable.

Each symbol used in the formula (I) is defined in the following.

In the present specification, “lower” means the presence of 1 to 6 carbon atom(s), unless otherwise specified.

Examples of the “lower alkyl” include straight chain or branched chain lower alkyl, such as methyl, ethyl, propyl, isopropyl, butyl, isobutyl, tert-butyl, pentyl, hexyl and the like, with preference given to methyl.

Examples of the “aryl” include phenyl, naphthyl, tolyl, xylyl, mesityl, cumenyl and the like, of which phenyl and naphthyl are preferable.

Examples of the “ar(lower)alkoxy” include benzyloxy, phenethyloxy, phenylpropoxy, benzhydryloxy, trityloxy and the like.

Examples of the “heterocyclic group” include a saturated or unsaturated monocyclic or polycyclic group having at least one heteroatom, such as nitrogen atom, oxygen atom, sulfur atom and the like.

Preferable examples of the above-mentioned “heterocyclic group” are a 3 to 8-membered, more preferably 5 or 6-membered, unsaturated heteromonocyclic group having 1 to 4 nitrogen atom(s), such as pyrrolyl, imidazolyl, pyrazolyl, pyridyl, pyridyl N-oxide, pyrimidyl, dihydropyridyl, tetrahydropyridyl, pyrazinyl, pyridazinyl, triazinyl, triazolyl, tetrazinyl, tetrazolyl and the like; an unsaturated fused heterocyclic group having 1 to 5 nitrogen atom(s), such as indolyl, isoindolyl, indolizinyl, benzimidazolyl, quinolyl, isoquinolyl, indazolyl, benzotriazolyl and the like; a 3 to 8-membered unsaturated heteromonocyclic group having 1 or 2 oxygen atom(s) and 1 to 3 nitrogen atom(s), such as oxazolyl, isoxazolyl, oxadiazolyl and the like; a 3 to 8-membered saturated heteromonocyclic group having 1 or 2 oxygen atom(s) and 1 to 3 nitrogen atom(s), such as morpholino, sydnonyl and the like; an unsaturated fused heterocyclic group having 1 or 2 oxygen atom(s) and 1 to 3 nitrogen atom(s), such as benzoxazolyl, benzoxadiazolyl and the like; a 3 to 8-membered unsaturated heteromonocyclic group having 1 or 2 sulfur atom(s) and 1 to 3 nitrogen atom(s), such as thiazolyl, isothiazolyl, thiadiazolyl and the like; a 3 to 8-membered unsaturated heteromonocyclic group having 1 or 2 sulfur atom(s), such as thienyl and the like; an unsaturated fused heterocyclic group having 1 or 2 sulfur atom(s) and 1 to 3 nitrogen atom(s), such as benzothiazolyl, benzothiadiazolyl and the like; a 3 to 8-membered unsaturated heteromonocyclic group having one oxygen atom, such as furyl and the like; an unsaturated fused heterocyclic group having 1 or 2 sulfur atom(s), such as benzothienyl and the like; and an unsaturated fused heterocyclic group having 1 or 2 oxygen atom(s), such as benzofuranyl and the like.

Examples of the “cyclo(lower)alkyl” include cyclopropyl, cyclobutyl, cyclopentyl and the like.

Examples of the “ar(lower)alkyl” include benzyl, phenethyl, phenylpropyl, benzhydryl, trityl and the like.

Examples of the “lower alkylene” include methylene, ethylene, propylene, pentamethylene, hexamethylene and the like.

The aforementioned lower alkyl, aryl, ar(lower)alkoxy, heterocyclic group, cyclo(lower)alkyl and ar(lower)alkyl are optionally substituted by a halogen atom (e.g., fluorine, chlorine, bromine and iodine).

While the brain disorder to be prevented or treated with a medicament containing, as an active ingredient, a compound having the above-mentioned potentiating effect is free of any particular limitation as long as it shows prophylaxis or alleviation of the disease state by potentiation of the N-type Ca²⁺ channel activity, the method of the present invention is particularly effective for the prophylaxis or treatment of dementia, amnesia, schizophrenia, manic-depressive psychosis, stroke, head trauma, nicotine withdrawal symptoms, spinal trauma, anxiety, thamuria, incontinence of urine, myotonic dystrophy, attention deficit hyperactivity disorder, narcolepsy, Parkinson's disease, autism and psychosomatic disorder.

An N-type Ca²⁺ channel activity-specific potentiator and an agent for the prophylaxis or treatment of brain disorders, which contains, as an active ingredient, a compound having an effect of specifically potentiating an N-type Ca²⁺ channel activity, to be used in the present invention (hereinafter the potentiator and the agent for prophylaxis/therapy are to be also collectively and simply referred to as “medicament used for the inventive method”), can be administered in a dosage form of a solid, semi-solid or liquid containing an organic or inorganic carrier or excipient, which is suitable for rectal administration, inhalation, nasal drop, eye drop, external (topical), oral and parenteral (inclusive of subcutaneous, intravenous and intramuscular) administrations and the like, direct administration to lesions of encephalon, spinal fluid, brain cavity and the like.

The medicament used for the inventive method can be admixed with a conventional, pharmaceutically acceptable and substantially non-toxic carrier or excipient, suitable for use for, for example, tablet, pellet, troche, capsule, suppository, cream, ointment, aerosol, powder for inhalation, liquid, emulsion, suspension and dosage forms suitable for other use. Moreover, auxiliary agent, stabilizer, thickener, colorant and flavor can be added as necessary.

The medicament used for the inventive method can be produced according to a technique known in the art for producing pharmaceutical preparations. The medicament used for the inventive method can be converted to a salt, prodrug or solvate, where necessary, according to a method known in the art.

In the present invention, the “salt” is preferably a biologically acceptable and generally non-toxic salt, which is, for example, acid addition salt such as acid addition salts with inorganic acid (e.g., hydrochloride, hydrobromide, sulfate, phosphate and the like), acid addition salts with organic carboxylic acid or sulfonic acid (e.g., formate, acetate, trifluoroacetate, maleate, tartrate, fumarate, methanesulfonate, benzenesulfonate, toluenesulfonate and the like), salts with acidic amino acid (e.g., aspartic acid, glutamic acid and the like) and the like.

In the present invention, the “prodrug” preferably means a compound that is converted to a compound having an effect of specifically potentiating an N-type Ca²⁺ channel activity, by reactions in the body with enzyme, gastric acid and the like.

In the present invention, the “solvate” is, for example, an inclusion compound (e.g., hydrate and the like).

The compound (I) to be used for the method of the present invention specifically potentiates an N-type Ca²⁺ channel activity in mammals such as human, monkey, mouse, rat, rabbit, pig, dog, horse, cow and the like, as well as animals such as birds, amphibian and the like, and is useful as a specific potentiator of N-type Ca²⁺ channel activity. Therefore, these can be the subjects of the method of the present invention. They can be the subjects of the method of the present invention not only at an individual level but also at a tissue or cell level. Even if the subject does not generally have an N-type Ca²⁺ channel, it can be the subject of the present invention upon artificial expression of the N-type Ca²⁺ channel.

When the medicament used for the inventive method is applied to an animal (inclusive of human), it is preferably administered intravenously (inclusive of infusion), intramuscularly or orally.

The medicament used for the inventive method only needs to be contained in a preparation in an amount sufficient to bring about a desired effect on the progression or conditions of the subject disease state.

While the dose and administration method of the medicament used for the inventive method vary depending on the kind of compound, age and condition of each patient undergoing the prophylaxis and/or treatment, it is 0.01-10 mg/kg body weight in the amount of compound (I), which is the active ingredient, per day for oral administration to patients. For the prophylaxis or treatment of the aforementioned diseases, the dose can be given at once or divided and given several times a day.

The present invention provides a method for screening a compound having an effect of specifically potentiating an N-type Ca²⁺ channel activity, which comprises measuring a cell membrane current, preferably a membrane current specific to each α₁ subunit of a neuronal voltage-dependent calcium channel.

In the present invention, the “neuronal voltage-dependent calcium channel α_{1B} subunit expression cell” is free of any particular limitation as long as it expresses a neuronal voltage-dependent calcium channel α_{1B} subunit, and may be naturally occurring or artificial. For example, a cell artificially made to express a neuronal voltage-dependent calcium channel α_{1B} subunit is a recombinant animal cell, which is specifically recombinant oocyte of Xenopus laevis obtained by removing follicle by a pretreatment with collagenase, and causing expression of α_{1B} subunit by injecting mRNA (e.g., commercially available products, polyA+RNA purified from fresh tissue, single cRNA synthesized using cloned cDNA as a template and cRNA synthesized from partial cDNA library) or cDNA into the nucleus, and the like.

In the present invention, the “neuronal voltage-dependent calcium channel α_{1B} non-expression cell” is free of any particular limitation as long as it does not express a neuronal voltage-dependent calcium channel α_{1B} subunit. It preferably expresses α₁ subunit other than α_{1B}, such as α_{1A}, α_{1E} and the like. These cells may be naturally occurring or artificial. Examples of the artificial cell include recombinant animal cells, which is specifically recombinant oocyte of Xenopus laevis obtained by removing follicle by a pretreatment with collagenase, and causing expression of a subunit other than α_{1B}, such as α_{1A}, α_{1E} and the like, by injecting the above-mentioned mRNA, and the like.

In the expression cell and/or non-expression cell, β subunit and α₂δ subunit, which are calcium channel-constituting subunits other than α₁ subunit, are preferably co-expressed to re-constitute a calcium channel.

In the present invention, the “test compound” is free of any particular limitation and any compound desired for determination if it has an effect of specifically potentiating an N-type Ca²⁺ channel activity can be used as a test subject. The method of the present invention is particularly suitable for screening a compound that may exhibit an effect similar to that of compound (I).

In the present invention, by the “bringing into contact” is meant a physical or chemical contact of the above-mentioned cell with a test compound. For example, the cell is suspended in a solvent, in which a test compound has been dissolved, or a test compound is dissolved in a solvent, in which the cell has been suspended, to achieve the contact of them. As such solvent, water, dimethyl sulfoxide (DMSO), artificial spinal fluid and the like can be exemplified.

The “membrane current” is a current flowing across a cell membrane, which is the total of a capacitative current that charges the membrane capacitance of the lipid bilayer of a biomembrane and an ion current flowing through an ion channel. In the present invention, Ca²⁺ channel alone is preferably used as the subject and a cell that expresses the channel alone is used. Therefore, an increase/decrease thereof in the membrane current in the present invention corresponds to an increase/decrease in the ion current, which is caused by the passage through a Ca²⁺ ion channel (the capacitative current is constant).

According to the present invention, the “measurement of the membrane current” is achieved by measuring a membrane current according to any method capable of evaluating changes in the membrane current. For example, two electrode membrane voltage-clamp method, Cut-Open method, patch-clamp method and the like are used. To be specific, two glass electrodes are immersed in a chamber filled with a recording liquid (divalent cation that passes through Ca²⁺ channel, such as Ba²⁺, Sr²⁺ and Ca²⁺) and a current is applied to allow measurement.

In one embodiment of the present invention, for example,
i) mRNAs of voltage-dependent calcium channels α_{1B}, α₂δ₁ and β_{1b} of rabbit are mixed and injected into Xenopus oocyte to prepare a neuronal voltage-dependent calcium channel α_{1B} subunit expression cell, the cell is brought into contact with a test compound and the membrane current of the cell is measured;
ii) mRNAs of voltage-dependent calcium channels α_{1A} or α_{1E}, and α₂δ₁ and β_{1b} of rabbit are mixed and injected into Xenopus oocyte to prepare a neuronal voltage-dependent calcium channel α_{1B} non-expression cell, the cell is brought into contact with a test compound and the membrane current of the cell is measured; and
iii) the membrane current of the expression cell and that of the non-expression cell are compared,
   whereby a compound having an effect of specifically potentiating an N-type Ca²⁺ channel activity can be screened.

The present invention is explained in detail in the following by way of Examples. These merely exemplarily show preferable embodiments of the present invention, and the present invention is not limited in any way by these Examples.

### EXAMPLES

Preparation of Oocyte

Ovary was removed from the abdominal cavity of Xenopus laevis and treated with collagenase to remove follicle covering the oocyte (Kinoshita et al., J. Biol. Chem. 276, 28731-28738 (2001)). A combination of three kinds of mRNAs (16.7 ng each) of voltage-dependent calcium channel subunits α_{1A}, α_{1B} or α_{1E} of rabbit and human α₂δ₁ and β_{1b} (prepared according to the method described in Kinoshita et al., supra or a similar method) was mixed and injected into the oocyte and this was incubated in modified Birth's saline (88 mM NaCl, 1 mM KCl, 0.41 mM CaCl₂, 0.33 mM Ca(NO₃)₂, 0.82 mM MgSO₄, 2.4 mM NaHCO₃, 7.5 mM Tris-HCl, 10 U/ml penicillin, 10 μg/ml streptomycin, pH 7.6) at 22° C. for 2 to 4 days.

Electrophysiological Measurement

The oocyte prepared in the above was placed in a chamber filled with a Ba²⁺-containing recording liquid (10 mM Ba(OH)₂, 90 mM NaOH, 2 mM KOH, 5 mM Hepes, 0.3 mM niflumic acid, methanesulfonic acid, pH 7.4) and fixed at −80 mV using two glass electrodes. A test pulse (200 ms width) toward 0 mV was applied at 30 second intervals and the whole-cell membrane current was measured. A +100 mV depolarization prepulse (100 ms width) that ends before 40 ms was applied every other pulse to eliminate the effect of G protein βγ subunit that binds with Ca²⁺ channel to suppress opening (Kinoshita et al., supra). Based on the Ba²⁺ current (I_{Ba}) as a Ca²⁺ channel current, the leak current was corrected by P/4 protocol. N-(4-Acetyl-1-piperazinyl)-p-fluorobenzamide monohydrate (hereinafter to be also referred to as a test compound) was dissolved in the recording liquid containing Ba²⁺ and applied.

<Results>

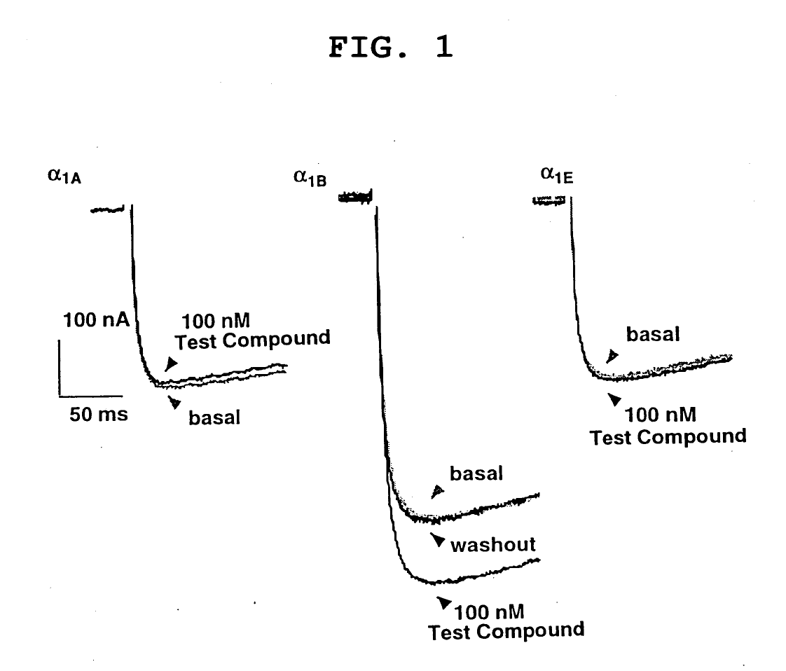

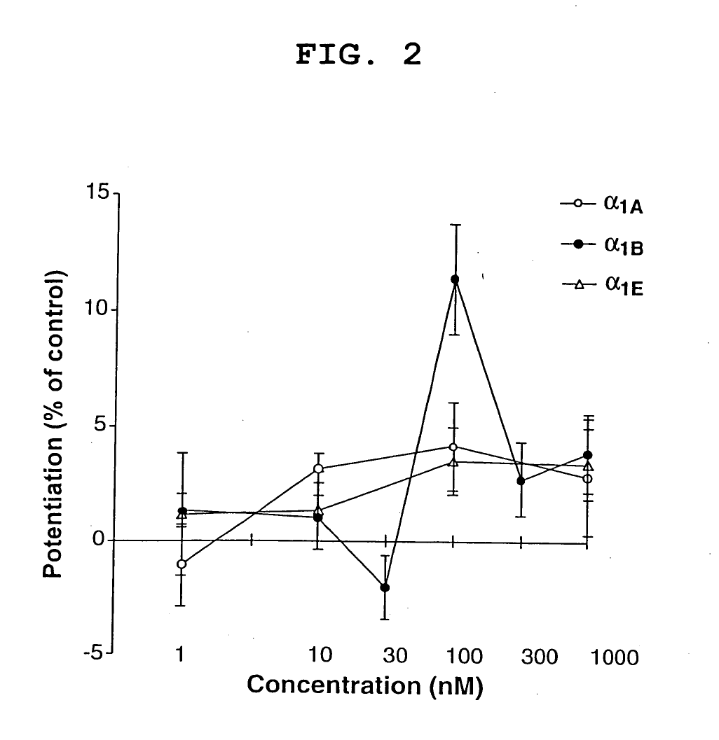

FIG. 1 shows the response upon application of 0 mV, 200 ms test pulse on the cells made to express any of the α_{1A}, α_{1B} and α_{1E} channels. When a 100 nM test compound was applied, the compound showed almost no effect on the cells made to express α_{1A} or α_{1E} channel. In contrast, the cell made to express α_{1B} channel showed an increase in the current. Soon after the cell was returned into a basal solution and washed, however, such action of the cell fell to the same level as that before the treatment. The effect of various concentrations of the test compound on the maximum amplitude of each channel was examined, the results of which are shown in FIG. 2.

For detailed investigation of the effect of the test compound on the α_{1B} channel, voltage-dependency with varied depolarization stimulating potential and voltage-dependency in steady inactive state were examined. A 100 nM test compound showed almost no influence on the voltage-dependency of α_{1B} channel activation/inactivation.

From these results, it has been clarified that the test compound scarcely exerts an influence on voltage-dependency of the α_{1B} channel and that the compound shows α_{1B} channel-specific potentiation of the current amplitude in a relatively narrow concentration range.

According to the present invention, an N-type Ca²⁺ channel activity-specific potentiator useful for the prophylaxis or treatment of brain disorders, such as dementia and amnesia, can be provided. In addition, the present invention provides a screening method of a compound having an effect of specifically potentiating an N-type Ca²⁺ channel activity, which is particularly useful for screening a compound having an effect similar to that of compound (I).

This application is based on patent application No. 2001-258808 filed in Japan, the contents of which are hereby incorporated by reference.

1-14. (canceled)

15. A method for screening a compound having an effect of specifically potentiating an N-type Ca²⁺ channel activity, comprising:
(a) contacting a cell expressing a neuronal voltage-dependent calcium channel α_{1B} subunit with a test compound; and measuring a membrane current of the cell;
(b) contacting a similar cell that does not express a neuronal voltage-dependent calcium channel α_{1B} subunit with a test compound; and measuring a membrane current of the cell;
comparing the currents measured in (a) and (b),
wherein a higher current value in (a) relative to (b) indicates a test compound which potentiates an N-type Ca²⁺ channel activity.

16. The method of claim 15, wherein the cells in (a) and (b) express the β subunit and α₂δ subunits.

17. The method of claim 15, wherein the cell that does not express a neuronal voltage-dependent calcium channel α_{1B} subunit, expresses an α₁ subunit other than the α_{1B} subunit.

18. The method of claim 15, wherein the cell expressing a neuronal voltage-dependent calcium channel α_{1B} subunit is a recombinant cell made to express said subunit.

19. The method of claim 15, wherein the cell expressing a neuronal voltage-dependent calcium channel α_{1B} subunit is an oocyte into which mRNA encoding the subunit is introduced.

20. The method of claim 15, wherein the cell expressing a neuronal voltage-dependent calcium channel α_{1B} subunit is a Xenopus oocyte into which mRNA encoding the subunit is introduced.

21. The method of claim 15, wherein the cell that does not express a neuronal voltage-dependent calcium channel α_{1B} subunit, expresses an α_{1A} or an α_{1E} subunit.

22. The method of claim 15, wherein the cell that does not express a neuronal voltage-dependent calcium channel α_{1B} subunit is a Xenopus oocyte into which mRNA encoding an α_{1A} or an α_{1E} subunit is introduced.
